(19) United States
(12) Patent Application Publication
(10) Pub. No.: US 2008/0033040 A1
(43) Pub. Date: Feb. 7, 2008

(54) Methods And Compositions For Screening Involving Endothelial Cells Under Shearing Conditions

(75) Inventors: Khalid Kader, Solon, IA (US); Christian H. Coyle, Greenock, PA (US)

Correspondence Address: FULBRIGHT & JAWORSKI L.L.P., 600 CONGRESS AVE., SUITE 2400, AUSTIN, TX, 78701, US

(73) Assignee: UNIVERSITY OF IOWA RESEARCH FOUNDATIN, IOWA CITY, IOWA (US)

(21) Appl. No.: 10/598,726

(22) PCT Filed: Mar. 9, 2005
(86) PCT No.: PCT/US05/07685

§ 371 Date: May 2, 2007

Related U.S. Application Data
(60) Provisional application No. 60/551,491, filed on Mar. 9, 2004.

(51) Int. Cl. A61K 31/21; A61K 31/19; A61P 3/10; A61P 9/00; C12N 5/00; C12Q 1/02; C12Q 1/26

(52) U.S. Cl. 514/506; 435/25; 435/29; 435/375; 514/564; 514/789

(57) ABSTRACT

The present invention concerns screening methods and compositions relating to endothelial cell function. More specifically, it provides screening methods to identify or characterize pathways and factors involved in endothelial cell function by employing conditions that simulate physiological stress. Additional methods can be implemented to identify candidate drugs for the treatment of endothelial cell dysfunction, such as cardiovascular disease or diabetes.

The present application claims priority to U.S. Provisional Patent Application No. 60/551,491, filed on Mar. 9, 2004, which is hereby incorporated by reference in its entirety.

### BACKGROUND OF THE INVENTION

1. Field of the Invention

The present invention relates generally to the field of cell biology. More particularly, it concerns methods and compositions for screening endothelial cells under conditions that mimic physiological stress conditions. In some embodiments, methods provide a way to identify or characterize agents or pathways in endothelial cell function. In other embodiments, methods of screening for candidate drugs to relieve oxidative stress in endothelial cells is provided. Thus, the present invention has ramifications for the fields of cardiology, diabetes, and other areas involving endothelial cells under oxidative stress.

2. Description of Related Art

Static culture studies of endothelial cells are the gold standard within vascular biology and tissue engineering. Because the physiology of endothelial cells under oxidative stress is not completely understood, it remains necessary to increase the abilities to probe endothelial cells in vitro (De Keulenaer et al., 1998; Fisher et al., 2001; Desai et al., 2002; Imberti et al., 2002; Nerem et al., 1998). This is especially true with regard to the upregulation of NO production by endothelial cells under physiologic shear (Corson et al., 1996) and the interplay between NO and SO. Intracellular measurements of both NO and SO are gauges of cellular function. Dysfunctional endothelial cells contribute to decreased levels of available nitric oxide due to heightened superoxide production (Jin et al., 2001; Tesfamariam et al., 1992; Tesfamariam, 1994).

Various researchers have commented on the need to include physiologic shear in studies of endothelial cells (Kader et al., 2000; Kader et al., 2002; Nerem, 1992; Muller-Glauser et al., 1988). However, the utility of the inclusion of physiologic shear has normally come into question due to the inability to use standard biological assays in the shearing systems. One example has been the reliable measurement of intracellular superoxide using dihydroethidium (DHE), a probe that diffuses freely through cellular membranes and is oxidized by superoxide, yielding ethidium bromide (Carter et al., 1994; Tarpey et al., 2001; Saiki et al., 1986) and causing a fluorescence shift from blue to red (Saiki et al., 1986) that can easily be detected by fluorescence microscopy and fluorimetry.

There remains a need for additional methods and processes to study endothelial cells so as to understand their physiology as well as to identify candidate drugs to relieve oxidative stress.

### SUMMARY OF THE INVENTION

The present invention is based on experiments showing that external factors can be applied vascular endothelial cells in shear studies as a viable way of obtaining meaningful information regarding the physiology and cell biology of such cells. Accordingly, the present invention concerns methods and compositions for evaluating vascular endothelial cells under conditions of physiologic stress. These methods and compositions can be employed to study vascular endothelial cells and elucidate pathways and factors involved in vascular endothelial cell function, particularly those that are involved when an endothelial cell is under stress. The present invention further provides methods and compositions for evaluating candidate substances for their ability to modulate vascular endothelial cells in a relevant physiological context.

Methods of the present invention include a method of identifying a protein or pathway involved in endothelial cell function. In specific embodiments, the invention is a method for testing whether an endothelial cell is under oxidative stress. In some embodiments, the method is implemented by (a) contacting an endothelial cell with (i) an indicator of a first cell function in the endothelial cell and (ii) a modulator of a known protein; (b) subjecting the endothelial cell to physiologic shear; and (c) assessing the first cell function, wherein a change in the first cell function in the presence of the modulator, as compared to the first cell function in the absence of the modulator, identifies the known protein as involved in the first cell function. It will be understood that “involved” means that the protein is a target or factor in a pathway involved in endothelial cell function. Specifically contemplated are those proteins involved in those aspects of endothelial function relating to disease states involving oxidation, particularly disease states involving superoxide. Thus, proteins that lead to, cause, are indicative of, or contribute to such disease states are of particular interest.

In methods of the invention, a particular protein or pathway may be modulated so that it is effectively inhibited. Under these conditions, levels of oxidative stress can be evaluated and/or determined to characterize the role or extent to which that particular protein or pathway is involved in endothelial cell function. Alternatively, such methods may serve to characterize or identify other proteins, factors, and/or pathways that are involved in endothelial cell function through the determination and/or evaluation of oxidative stress. In these instances, an indicator such as DHE may be employed.

Methods of the invention also include methods for screening for a candidate drug to relieve or reduce oxidative stress in an endothelial cell. In some embodiments, methods involve one or more of the following steps: (1) contacting an endothelial cell with DHE; (2) contacting the endothelial cell with a candidate substance; (3) contacting the endothelial cells with hydrogen peroxide; (4) subjecting the endothelial cell to physiologic shear; (5) comparing the amount of DHE oxidized by the cell with the amount of DHE oxidized by a cell not contacted with the candidate substance; (6) measuring the amount of DHE taken up by the cell; and (7) assaying or evaluating fluorescence from the cell. A difference in amounts of DHE oxidized indicates the candidate substance is a candidate drug, particularly in cases where the amount of DHE oxidized is lower than a control cell or sample. Fluorescence may be detected by any method known to those of skill in the art including, but not limited to, fluorescence microscopy and fluorimetry.

It is contemplated that the candidate substance is, in some embodiments, a protein, nucleic acid, or small molecule, and it may be known or unknown. The protein may be an antibody (monoclonal, polyclonal, single chain or humanized), a chimeric or fusion protein, dominant negative protein, a truncated protein or a full-length protein. The nucleic acid may be RNA or DNA, and it may be single or double stranded. It is contemplated that a nucleic acid may encode a polypeptide or it may be an antisense molecule, an siRNA, an miRNA, or other noncoding nucleic acid molecule. Small molecule candidate substances may be part of a combinatorial library or may be individual substance that may or may not mimic natural substances involved in endothelial cell function. It is contemplated that the candidate substance may be a modulator of endothelial cell function, as described herein.

It is contemplated that the amount of DHE oxidized by the cell in the assay may be compared to a standardized amount of DHE oxidized in a cell under similar conditions but in the absence of the candidate substance; thus, in some embodiments, a control sample is evaluated alongside the cell in question, while in other embodiments, a standardized amount or a control sample is simply referred to and not evaluated. It is contemplated that candidate substances can be identified as candidate drugs in virtue of their detected ability to reduce or relieve the amount of superoxide in the endothelial cell.

Further methods of the invention include contacting the cell with an inhibitor of eNOS, NADPH, endothelial nitric oxide synthase, NADH/NADPH-oxidase, cyclooxygenase, or xanthine oxidase. The effect of a candidate substance under these circumstances can also be used to evaluate its potential efficacy.

The amount of an indicator that an endothelial cells is contacted with or exposed to is about, about at least, or about at most 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 μM/ml or more of the indicator, or any range derivable therein. In some cases, between about 1 μM and about 5 μM of DHE is employed in method of the invention. In specific embodiments, about 2.0 μM of DHE is used.

Furthermore, it is contemplated that an endothelial cell from an animal or organism suffering from a disease state involving oxidation may be used in methods of the invention. Alternatively, an endothelial cell with a genotype that is indicative of a disease state involving oxidation may also be used.

After this in vitro evaluation, subsequent evaluations may involve an animal model or other in vivo testing, as discussed below.

It is contemplated that in embodiments of the invention, the endothelial cell is contacted with the indicator before, at the same time, and/or after the cell is subjected to physiologic shear conditions and the effect of shearing on the cell is evaluated by evaluating the indicator or the amount of indicator.

In some embodiments of the invention, the protein is an enzyme, receptor, transporter signaling molecule, or transcription factor. In certain embodiments, the first cell function is nitric oxide (NO) production, regulation of intracellular superoxide (SO) levels, and/or superoxide dismutase activity. In additional embodiments, intracellular SO levels involves the following pathways: endothelial nitric oxide synthase, NADH/NADPH-oxidase, and/or cyclooxygenase, xanthine oxidase.

Methods of the invention involve indicators of cell function. The term “indicator” is used according to its plain and ordinary scientific meaning to refer to a substance that indicates the presence of another substance or quality (see Oxford English Dictionary). It can also be a “quantitative indicator,” meaning it indicates the quantity of another substance or this other substance's activity, in addition to indicating the presence of the other substance. It is contemplated that the indicator may be, in some embodiments of the invention, a protein, nucleic acid, and/or small molecule, and that the indicator is enzymatic, colorimetric, radioactive, or fluorescent. Moreover, in some embodiments, the indicator is specifically contemplated as not having certain of those functional or structural characteristics; for example, the indicator, in some embodiments is a non-proteinaceous indicator, meaning it is an indicator that does not contain any protein or is not a protein. It is specifically contemplated that the indicator may be a non-enzymatic indicator, which means the indicator does not have enzymatic activity. In additional embodiments, the indicator is dihydroethidium (DHE). Other indicators may be cytochrome c (reduction), as well as lucigenin or coelenterazine (chemiluminescence). Such indicators can be used, in some embodiments, to evaluate cell functions concerning oxidative stress under physiologic shearing conditions.

Endothelial cells are exposed to shearing conditions to simulate physiological conditions. Cells are placed under shear in methods of the invention. Such conditions are well known to those of skill in the art and the apparatus for generating such conditions are readily and commercially available. Exemplary conditions include, but are not limited to, exposing the cells to a constant flow rate of about, about at least, or about at most 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, 28.0, 28.1, 28.2, 28.3, 28.4, 28.5, 28.6, 28.7, 28.8, 28.9, 29.0, 29.1, 29.2, 29.3, 29.4, 29.5, 29.6, 29.7, 29.8, 29.9, 30.0, 30.1, 30.2, 30.3, 30.4, 30.5, 30.6, 30.7, 30.8, 30.9 or more ml/min (or the equivalent in dynes/cm²), or any range derivable therein. Alternatively, cells may be exposed to shearing conditions of about, at least about, or at most about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 24.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0 or more dynes/cm², or any range derivable therein.

The cells may be exposed to shearing conditions for 5, 10, 15, 20, 25, 30, 35, 40, 45, 50, 55, 60, minutes and/or 1, 2, 3, 4, 5, 6, 7, 8, 9, 10, 11, 12 or more hours, or any range derivable therein. In specific embodiments, cells are exposed to a flow rate of 24.4 ml/min (2.2 dynes/cm²) for 1.5 hours. It is specifically contemplated that in some embodiments, the cell is contacted with the inhibitor prior to undergoing shear conditions.

In addition to exposing an endothelial cell to shearing conditions, the cell can also be exposed to other agents or conditions that alters the cell physiology. In some embodiments, oxidative stress is induced in the cell. Oxidative stress can be induced by exposing the cell to hydrogen peroxide (H₂O₂). In some embodiments, cells are exposed to about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 200, 300, 400, 500, 600, 700, 800, 900, 1000 or more μM, or ranges derivable therein, of the agent. In particular embodiments, an endothelial cell is contacted with about 30 μM to about 100 μM of hydrogen peroxide. In some cases, a cell is contacted with about 30 μM of hydrogen peroxide.

Methods of the invention involve contacting endothelial cells with a modulator protein involved in endothelial cell function. The term “modulator” is used according to its plain and ordinary scientific meaning, and it refers to a substance that exerts a modifying or controlling influence on or regulates an endothelial cell function (see Oxford English Dictionary). Accordingly, modulators may inhibit, reduce, eliminate, enhance, promote, induce, or increase a particular cell function. It is contemplated that in some embodiments, modulators are limited as such—for example, the modulator may inhibit cell function, in which case it is an inhibitor of cell function. In specific embodiments, the modulator inhibits an enzyme involved in endothelial cell function, particularly SO production.

Modulators may be proteins, nucleic acids, or small molecules, or they may be designated as specifically not those substances. For example, the modulator may be a non-proteinaceous modulator, meaning the modulator does not contain protein or is not a protein. In some embodiments, the modulator is selected from the group consisting of an agonist of a cell function, an antagonist of cell function, an agent that acts on an enzyme in cell function, an agent that acts on a receptor in cell function, an agent that acts on a signaling molecule in cell function, an agent that acts on a transporter in cell function, or an agent that acts on a transcription factor in cell function. In specific embodiments, the modulator is an antagonist of eNOS, nicotinamide adenine dinucleotide phosphate (NADPH), cyclooxigenase, xanthine oxidase, lipooxygenase, or NADH-oxidase. In some embodiments, the modulator may be an antagonist of a receptor, transporter, enzyme, or transcription factor.

In some embodiments, methods include manufacturing a candidate compound. The manufacturing can include one or more steps that allow the compound to be administered to an organism or to biological matter. For example, the manufacturing can include an purification or isolation step. Another example is that the manufacturing can involve a formulation step in which the compound is formulated for administration to an organism or biological matter. In certain embodiments, the compound is administered to an animal or patient.

In other embodiments, methods include further testing a candidate compound in an animal model or in a patient (such as in a clinical trial). Testing of a compound may involve simply determining that it is of a certain purity or has a particular level of activity. Such steps may be viewed as quality control implementations.

The present invention also concerns methods of inhibiting NO production in an endothelial cell. In some embodiments, a method involves contacting an endothelial cell with an effective amount of an inhibitor of eNOS, NADPH, cyclooxigenase, xanthine oxidase, lipooxygenase, or NADH-oxidase. In some embodiments, the inhibitor is L-NAME, L-NNA, flurbiprofen, or allopurinol. Other inhibitors are well known to those of skill in the art and they can be used in methods of the invention.

In methods of the invention, it is contemplated that an endothelial cell may be in cell culture or it may be located in a living organism or animal. It is contemplated that screening methods of the invention may first be performed in vitro but that additional testing is done in vivo. In the latter case, the endothelial cell is contacted with the modulator, indicator, inhibitor, or candidate drug by administering an effective amount of it to the organism.

In some embodiments, the organism or animal is a mammal, which includes, but is not limited to, humans, monkeys, mice, rats, pigs, rabbits, dogs, and cats. It is contemplated that the organism may have a disease or condition that involves endothelial dysfunction. Such conditions and diseases include diabetes mellitus, cardiovascular diseases (such as ischaemic heart disease, angina pectoris, myocardial infarction, congestive heart failure, atherosclerosis, hypertension and arrhythmia), asthma, trauma, shock (hypovolumic, neurogenic or septic), neurotoxicity, neurodegenerative and neurological disorders (including Alzheimer and Parkinson's diseases, amyotrophic lateral sclerosis, multiple sclerosis, convulsive (seizure) disorders, AIDS-dementia and disorders that involve processes of learning and memory), disorders of gastric secretions, relaxation and peristalsis of the intestinal tract (including sphincters), drug and disease-induced nephropathies, pathological (premature) and physiological uterine contractions, cellular defense impairment, endothelial dysfunction-induced diseases and insulin-resistance in diabetes, pregnancy-induced hypertension, chemotaxis and phagocytic impairment in immunological disorders, cerebrovascular diseases, aggregation disorders, fertility and reproductive disorders (e.g., penile erection and treatment of male impotence). In some embodiments, the disease or condition is diabetes or cardiovascular disease.

The present invention also concerns methods of treating chronic oxidative stress in an animal comprising administering to an endothelial cell in the animal an effective amount of an inhibitor of eNOS, NADPH, cyclooxigenase, xanthine oxidase, lipooxygenase, or NADH-oxidase. Inhibitors include, but are not limited to, L-NAME and L-NNA, as well as flurbiprofen or allopurinol. It is contemplated that an effective amount of the inhibitor achieves a therapeutic benefit for the animal with respect to the oxidative stress. It is contemplated that the oxidative stress in the cell is relieved, reduced, ameliorated, and/or eliminated, which may be determined by indirect or direct assays of oxidative stress. Such assays are well known to those of skill in the art.

It is contemplated that any method or composition described herein can be implemented with respect to any other method or composition described herein.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF ILLUSTRATIVE EMBODIMENTS

The current study demonstrates the ability of hydrogen peroxide to activate vascular endothelial cells resulting in endothelial cell superoxide production. The superoxide produced by the endothelial cells is shown to lead to cellular dysfunction under physiological shear and static culture. Superoxide production was further isolated to its enzymatic sources, with the discovery of uncoupled eNOS as a significant contributor to superoxide production. These results show that hydrogen peroxide stimulation of endothelial cells leads to eNOS-dependant superoxide production, which indicates that the reaction of endothelial cells to H₂O₂ exposure follows a different pathway than that of vascular smooth muscle cells. This may provide another therapeutic target for the reduction of chronic oxidative stress.

Moreover, the Examples show that endothelial cells can be evaluated in shear studies in the presence of an external marker to assay and evaluate intracellular function, such as superoxide production.

### I. Endothelial Cells and Oxidative Stress

Hypertension and diabetes are major factors with regard to the development and progression of cardiovascular disease (American Heart Association, 2003). Both lead to endothelial cell dysfunction and damage (Cai et al., 2000; Strawn et al., 2002; Tesfamariam et al., 1992; Tesfamariam, 1994), as well as proliferation and activation of smooth muscle cells (Strawn et al., 2002; Sodhi et al., 2003).

Consequently, cellular level studies of disease states such as diabetes and hypertension require utilization of endothelial cells. Endothelial cells regulate and maintain the chemical balance of the cardiovascular system and surrounding tissues. In hypertension and diabetes, oxidative stress is heightened and decreases the availability of endothelial cell factors such as nitric oxide (Vanizor et al., 2001; Jeremy et al., 2002; McQuaid et al., 1997). Within an oxidative environment, the angiotensin II type 1A endothelial cell surface receptor is upregulated (Ullian et al., 1997; Schiffrin et al., 2002), leading to increases in multiple factors including platelet derived growth factor (PDGF) (Tazawa et al., 1999), transforming growth factor β1 (TGF-β1), and nuclear factor κB (NF-κB) (Schiffrin et al., 2002; Cowling et al., 2002). This leads to aberrant smooth muscle cell activity and further loss of endothelial cell function. The loss of endothelial cell function leads to a reduction in available nitric oxide (NO) and superoxide dismutase (SOD) production (Jin et al., 2001), with a corresponding increase in intracellular superoxide (SO) concentrations. The increase in SO concentration leads to enhancement of peroxynitrite formation and DNA oxidation and cell damage (Jin et al., 2001; Beckman et al., 1990).

The effects of reactive oxygen species-mediated oxidative stress on tissue damage are well established. Hydrogen peroxide has been shown to activate various vascular cells including smooth muscle cells (Baas et al., 1995; Rao et al., 1993; Gonzalez-Pacheco et al., 2002; Rao et al., 1993), cardiomyocytes (Chandrasekar et al., 2003; Von Harsdorf et al., 1999), and microvascular endothelial cells (Cai et al., 2000; True et al., 2000) to produce superoxide (O₂—). Recently, it was found that H₂O₂ triggers NADPH-oxidase dependant superoxide production from vascular smooth muscle cells (Li et al., 2001). H₂O₂ has also been found to stimulate eNOS and nitric oxide production. Superoxide has been shown to be a potent oxidative agent through its ability to form peroxynitrite through combination with nitric oxide, thus reducing the level of free NO (Beckman et al., 1990; Kojda et al., 1999; Pryor et al., 1995).

The precise mechanisms and pathways involved in superoxide production as it relates to disease states are not, however, fully understood. Thus, additional approaches to studying endothelial cells under stress conditions are needed.

### II. Screening Methods

It will, of course, be understood that all the screening methods of the present invention are useful in themselves as a way of understanding cell function, notwithstanding the fact that effective candidates may not be found with respect to the treatment of oxidative stress in endothelial cells. The invention provides methods for identifying factors involved in endothelial cell function, particularly those cell function relating to oxidative stress.

Methods of the invention provide for a number of ways to place endothelial cells under stress conditions and these include both internally and externally applied conditions. Internally applied conditions are those involving one or more agents that are internalized by the cell, such as a small molecule, protein, or nucleic acid. Embodiments of the invention involve the use of hydrogen peroxide, for example. Externally applied conditions refer to shearing conditions. Such conditions can be readily simulated using commercially available machinery and apparatuses. Furthermore, improvements in this technology are also being developed, and these are contemplated for use with the invention as well (Blackman et al., 2000), which is hereby incorporated by reference.

A. Modulators

As used herein the term a “modulator” refers to a compound or substance that affects activity, directly or indirectly, of a particular molecule. A modulator may be an “inhibitor,” which is a compound that overall effects an inhibition of activity with respect to the particular molecule; this may be accomplished by inhibiting the molecule's expression, translocation or transport, function, expression, post-translational modification, location, half-life, or more directly by preventing its activity, such as by binding the molecule. A modulator may be an “enhancer,” which enhances or increases the molecule's activity, by increasing, for example, expression, translocation or transport, function, expression, post-translational modification, location, half-life, or more directly its activity. An direct increase in the molecule's activity may be accomplished by increasing, for example, its binding activity. Any modulator described in methods and compositions herein may be an inhibitor or an enhancer. Similarly, modulators may be “agonists” or “antagonists,” which are terms used according to their ordinary and plain meanings.

A “candidate substance” refers to any molecule that exhibits one or more desirable characteristics with respect a screening method. The candidate substance may be a protein or fragment thereof, a small molecule, or even a nucleic acid molecule. Using lead compounds to help develop improved compounds is know as “rational drug design” and includes not only comparisons with known inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate substances may include fragments or parts of naturally-occurring compounds, or may be found as active combinations of known compounds, which are otherwise inactive. It is proposed that substances isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are well known to those of skill in the art. For example, an antisense molecule that bound to a translational or transcriptional start site, or splice junctions, would be ideal candidate inhibitors.

In addition to the modulating substances initially identified, the inventors also contemplate that other sterically similar compounds may be formulated to mimic the key portions of the structure of the modulators. Such substances, which may include peptidomimetics of peptide modulators, may be used in the same manner as the initial modulators.

An inhibitor or activator according to the present invention may be one which exerts its inhibitory or activating effect upstream, downstream or directly on superoxide production or nitric oxide production. Regardless of the type of inhibitor or activator identified by the present screening methods, the effect of the inhibition or activator by such a compound results in alteration in the amount of stress a cell is under as compared to that observed in the absence of the added candidate substance.

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

Also, indicators are employed in some methods of the invention. They provide a way to measure a modulator's effect on cell function or the effect of a candidate substance with respect to level of oxidative stress.

A technique for high throughput screening of compounds is described in WO 84/03564.

Assays may be conducted in cell free systems, in isolated cells, or in organisms including transgenic animals.

Indicators, modulators, and candidate substances of the present invention may be different and varied compounds. These include enzymes, drugs (e.g., antibacterial, antifungal, anti-viral), antibody regions, regions that mediate protein-protein or ligand-receptor interactions, cytokines, growth factors, transporters, receptors, signaling molecules, hormones, toxins, polynucleotides coding for proteins, antisense sequences, radiotherapeutics, chemotherapeutics, ribozymes, tumor suppressors, transcription factors, inducers of apoptosis, or liposomes containing any of the foregoing. In addition to encompassing the delivery of purified compounds, the present invention further contemplates the delivery of nucleic acids that encode cognate compounds such as polypeptides. Therefore, according to the present invention, both purified compounds and nucleic acid sequences encoding that compound, e.g., a cytokine, may be involved in screening methods of the invention.

### III. Therapeutic Methods and Compositions

In an embodiment of the present invention, a method of relieving oxidative stress or treating endothelial dysfunction are contemplated. Such conditions and diseases include diabetes mellitus, cardiovascular diseases (such as ischaemic heart disease, angina pectoris, myocardial infarction, congestive heart failure, atherosclerosis, hypertension and arrhythmia), asthma, trauma, shock (hypovolumic, neurogenic or septic), neurotoxicity, neurodegenerative and neurological disorders (including Alzheimer and Parkinson's diseases, amyotrophic lateral sclerosis, multiple sclerosis, convulsive (seizure) disorders, AIDS-dementia and disorders that involve processes of learning and memory), disorders of gastric secretions, relaxation and peristalsis of the intestinal tract (including sphincters), drug and disease-induced nephropathies, pathological (premature) and physiological uterine contractions, cellular defense impairment, endothelial dysfunction-induced diseases and insulin-resistance in diabetes, pregnancy-induced hypertension, chemotaxis and phagocytic impairment in immunological disorders, cerebrovascular diseases, aggregation disorders, fertility and reproductive disorders (e.g., penile erection and treatment of male impotence).

An effective amount of the pharmaceutical composition, generally, is defined as that amount sufficient to detectably and repeatedly to ameliorate, reduce, minimize or limit the extent of the disease or its symptoms. More rigorous definitions may apply, including elimination, eradication or cure of disease.

A. Administration

To relieve oxidative stress and/or treat endothelial dysfunction using the methods and compositions of the present invention, one would generally contact an endothelial cell with the therapeutic compound, which may be identified in screening methods of the invention. The routes of administration will vary, naturally, with the nature of the condition or disease, and include, e.g., intradermal, transdermal, parenteral, intravenous, intramuscular, intranasal, subcutaneous, percutaneous, intraperitoneal, perfusion, lavage, and oral administration and formulation. A stent may be used to administer the compound.

To effect a therapeutic benefit with respect to endothelial dysfunction, one would contact a vascular cell with the therapeutic compound. Any of the formulations and routes of administration discussed with respect to the treatment or diagnosis of cancer may also be employed with respect to vascular diseases and conditions.

Administration may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

Treatment regimens may vary as well, and often depend on tumor type, tumor location, disease progression, and health and age of the patient. Obviously, certain types of tumor will require more aggressive treatment, while at the same time, certain patients cannot tolerate more taxing protocols. The clinician will be best suited to make such decisions based on the known efficacy and toxicity (if any) of the therapeutic formulations.

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

Dosage levels that are used may vary over quite a wide range depending upon the compound used, the severity of the symptoms exhibited by the patient and the patient's body weight. Without limitation to the present invention, typical dosages, for example for the treatment of angina, may be about 1 to about 100 mg, particularly about 5 to about 40 mg, given two or three times daily or about 1 to about 200 mg, including about 20 to about 50 mg, in sustained release formulations given once or twice daily. Typical dosages, for example for acute myocardial infarction, may be about 0.1 to about 10 mg, preferably about 1 to about 2 mg, sublingually; about 0.5 to about 50 mg, preferably about 5 to about 10 mg, orally; or about 1 to about 100 micrograms, preferably about 10 to about 20 micrograms, per minute intravenously.

B. Combination Treatments

The compounds and methods of the present invention may be used in the context of endothelial dysfunction. In order to increase the effectiveness of a treatment with the compositions of the present invention, it may be desirable to combine an agent identified in screening methods of the invention with other agents that have been traditionally effective in the treatment of those diseases and conditions. For example, the treatment of cardiovascular disease may be implemented with therapeutic compounds of the present invention and other cardiovascular therapies, such as hypertensive drugs or angioplasty. Likewise, the treatment of a diabetic disease or condition may involve both an agent identified in a screening method of the invention and conventional diabetes therapies, such as insulin.

Various combinations of agents may be employed; for example, a candidate agent identified in a screening method of the invention is “A” and the secondary agent/therapy is “B”:

A/B/A B/A/B B/B/A A/A/B A/B/B B/A/A A/B/B/B
B/A/B/B B/B/B/A B/B/A/B A/A/B/B A/B/A/B
A/B/B/A B/B/A/A B/A/B/A B/A/A/B A/A/A/B
B/A/A/A A/B/A/A A/A/B/A

Administration of the therapeutic expression constructs of the present invention to a patient will follow general protocols for the administration of that particular secondary therapy, taking into account the toxicity, if any, of the combination treatment. It is expected that the treatment cycles would be repeated as necessary. It also is contemplated that various standard therapies, as well as surgical intervention, may be applied in combination with the described endothelial cell dysfunction.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Standardization of Intracellular Superoxide Determinations by Dihydroethidium Under Physiological Shear

### Materials and Methods

1. Cell Culture

Porcine aortic endothelial cells (PAEC) were cultured in Medium 199 (M199, Invitrogen, Grand Island, N.Y.) supplemented with 1% penicillin-streptomycin (Invitrogen, Grand Island, N.Y.) and 10% fetal bovine serum (FBS, Hyclone, Logan, Utah). PAEC were incubated at 37° C. with 95% humidity and 5% CO₂. For static experimentation, endothelial cells were cultured and plated into 24-well plates at a density of 37,000 cells/well. For experiments under shear, PAEC were cultured and plated at a density of 500,000 cells/slide onto glass slides (1″×3″, Surgipath, Richmond, Ill.), coated with Vitrogen (0.032 mg/ml, Cohesion, Palo Alto, Calif.). In all experiments, PAEC were used from passage 5 through passage 10. Under shear, PAEC were cultured in circulating medium consisting of serum-free, phenyl-red-free M199.

2. Optimization of H₂O₂ Concentration

Hydrogen peroxide has previously been shown to increase intracellular superoxide levels in vascular cells (Carter et al., 1994; Li et al., 2001). These levels of superoxide are commonly measured using dihydroethidium and other methods such as reduction of cytochrome c (Boveris et al., 2002), and the chemiluminescence of lucigenin (Hohler et al., 2000) or coelenterazine (Tarpey et al., 1999).

PAEC were maintained under 1.5 hour exposure to hydrogen peroxide as well as without exposure as a negative control. Under static conditions, four different concentrations of hydrogen peroxide (3 μM, 30 μM, 100 μM, and 1 mM) were used to optimize H₂O₂ concentration. These concentrations are consistent with published concentrations of H₂O₂ used during in vitro experimentation which vary between 60-200 μM.

Testing under shear was conducted using an Immunetics flow cell (Immunetics, Cambridge, Mass.) (Li et al., 1996). This was incorporated into a flow system including a temperature controlled reservoir (Precision, Winchester, Va.) and cyclical cartridge pump (Ismatec SA, Chicago, Ill.; FIG. 1). The flow system provided for oscillatory, laminar flow across the sample. PAEC were visualized under flow using our image analysis system consisting of a Zeiss Axiovert S100 and AxioCam MR digital camera (Carl Zeiss, Thornwood, N.Y.). An increase in hydrogen peroxide concentration was necessary to counter the additional degradation of hydrogen peroxide due to constant light exposure due to microscopy and free radical scavenging by the polycarbonate cell adhesion chamber and tubing. Due to these factors, an increase in concentration to 100 μM H₂O₂ was necessary for experimentation under shearing conditions.

3. Establishing the Use Dihydroethidium Under Static Conditions

PAEC were incubated for one hour in serum-free M199 with 30 μM H₂O₂; non-exposed PAEC were incubated in serum-free M199. At one hour after the start of incubation, 2.0 μM dihydroethidium was added to each well. An additional 30 minutes of incubation was allowed. PAEC were then washed and cultured in phenyl red free and serum free M199. Fluorescent images were captured using our imaging system under a fluorescein filter (FITC) at an exposure of 4500 ms.

4. Establishing the Use Dihydroethidium Under Shearing Conditions

An Immunetics flow cell (Li et al., 1996) was prepared by first flushing the fluid path with deionized water and then phenyl red free M199. After flushing, 50 ml of the circulating medium was added and allowed to perfuse for 5 minutes while warming to 37° C. PAEC were then placed in the flow cell and the flow cell was secured to the microscope stage. Endothelial cells were maintained at 37° C. by fluid flow through a water bath (Precision, Winchester, Va.), at a flow rate of 24.4 ml/min (2.2 dynes/cm2), for 1.5 hours under 100 μM H₂O₂ in circulating medium. The PAEC not exposed to H₂O₂ were subjected to the same flow rate in serum free and phenyl red free M199 without H₂O₂. After 1 hour of incubation, 2.0 μM dihydroethidium was added to the fluid reservoir. The experiment was conducted under constant imaging, with images being captured at 0, 15, 30, 60, and 90 minutes. A fluorescent image was also taken at 90 minutes using a fluorescein filter (FITC) at an exposure of 4500 ms. All fluorescent images were acquired with phenyl red free media to eliminate interference of phenyl red and FBS in fluorescent imaging.

### Results

### H₂O₂ Optimization

Initial experiments were conducted at 3 μM, the heightened level of H₂O₂ found in cardiovascular diseases (Lacy et al., 1998). At 3 μM H₂O₂; 1.2±0.2% of PAEC exposed to H₂O₂ demonstrated intracellular superoxide, while 1.1±0.2% of non-exposed PAEC were found to produce superoxide. At this concentration of hydrogen peroxide, no statistically significant increase in intracellular superoxide concentration was observed between H₂O₂ exposed and non-exposed PAEC.

An increase to 30 μM H₂O₂ achieved a decipherable signal in static experiments. At this concentration, 77.1±5.5% (p<0.001) PAEC were found to produce intracellular superoxide, while 4.0±0.9% of non-exposed PAEC were found to produce superoxide. While this concentration is high with respect to that found in the body, it may be necessary in order to compensate for free radical scavenging by the tissue culture polystyrene plates used. Again, this concentration is lower than most other published studies. Increasing H₂O₂ concentration beyond the 30 μM did not lead to an increase in the number of endothelial cells showing intracellular superoxide stress. However, PAEC exhibited loss in cell membrane integrity in a dose dependant manner with increases in hydrogen peroxide concentration, most notably at 1 mM. At 1 mM, 76.1±6.8% PAEC exhibited fluorescent nuclei compared to 1.6±0.3% of non-exposed PAEC. No noticeable increase was observed in the percentage of PAEC exhibiting increased intracellular superoxide concentration, though the majority of PAEC in 1 mM experiments lysed during experimentation.

### Establishing the Use Dihydroethidium Under Shearing Conditions

Experiments were conducted at 100 μM H₂O₂ under shear. At 100 μM H₂O₂, 49.5±3.3% of PAEC exposed to H₂O₂ demonstrated intracellular superoxide, while 1.8±0.8% of non-exposed PAEC exhibited superoxide stress. At this concentration of hydrogen peroxide, a statistically significant (p<0.001) increase in intracellular superoxide concentration was observed between H₂O₂ exposed and non-exposed PAEC. Further, there is a statistically significant difference between endothelial cells tested under shearing and static conditions (p<0.001).

### Example 2

### MnSOD Over-Expressing Endothelial Cells Reduce Oxidative Stress in Both Static and Shearing Conditions

### Materials and Methods

1. Cell Culture

PAEC were cultured in Medium 199 (Invitrogen, M199) supplemented with 1% Penicillin-Streptomycin (Invitrogen) and 10% Fetal Bovine Serum (Hyclone, FBS) and maintained at 37° C. with 95% humidity and 5% CO₂. For experimentation, PAEC were cultured and plated into 24-well plates, at a density of 37,000 cells/ml. PAEC were used from passage 5 through passage 10 in all experiments.

2. Adenoviral Infection of PAEC

Adenoviral-mediated gene transfer was conducted by infection of endothelial cells (85-95% confluence, University of Iowa Cardiovascular Research Center Cell Culture Facility) as previously described (Fang et al., 1998). Ad5CMVSOD2, Ad5CMVeNOS, and Ad5CMVeGFP were acquired from the University of Iowa Gene Transfer Vector Core (REF); Ad5CMVeGFP was used as a negative control. Porcine aortic endothelial cells were incubated with 125 PFU/cell MnSOD/eNOS/eGFP adenovirus in serum-free M199. To improve the efficiency of adenoviral uptake, the adenoviral stock was incubated for 30 minutes prior to addition to cell cultures (Kossila et al., 2002). Infected PAEC were incubated under standard conditions. After 3 hours, the virus-containing solution was removed, samples were washed with serum-free M199, and M199 supplemented with 1% Penicillin-Streptomycin and 2% FBS was added. After 45 hours of incubation (48 hours after initiation of infection) cells were prepared for experimentation.

3. Determination of Intracellular Superoxide

PAEC were incubated for 1 hour in 60 μM H₂O₂ (serum-free M199); control PAEC were incubated in serum-free M199. Incubation continued for an additional 30 minutes with 2 μM dihydroethidium (Miller et al., 1998; Carter et al., 1994) (DHE, Molecular Probes). After incubation, samples were washed and cultured in phenyl-red-free and serum-free M199. Images were captured using a Zeiss Axiovert S100 fluorescent microscope. Images were collected at a standard exposure of 4500 ms.

4. L-NAME Inhibition of eNOS

In order to ascertain the role of eNOS in endothelial cell superoxide production, the presence of superoxide was probed in the presence of L-NAME. PAEC were incubated for 1 hour with 1 mM N^{G}-nitro-L-arginine methyl ester (Kader et al., 2000) (Sigma, L-NAME) in serum-free M199; control groups were incubated in serum-free M199; samples in both control and experimental groups were also exposed to 60 μM H₂O₂. After incubation, 2 μM DHE was added to each sample and incubated for 30 minutes. After incubation, PAEC were washed with serum-free M199 and imaged at an exposure of 2500 ms.

5. Determination of eNOS Couple State

In order to ascertain the role of the coupled state of eNOS in endothelial cell superoxide production, the presence of superoxide was probed in the presence of ascorbic acid. PAEC were incubated for 1 hour with 1 mM Ascorbic Acid (CalBiochem) in serum-free M199; control groups were incubated in serum-free M199; samples in both control and experimental groups were also exposed to 60 μM H₂O₂. After incubation, 2 μM DHE was added to each sample and incubated for 30 minutes. After incubation, PAEC were washed with serum-free M199 and imaged at an exposure of 1200 ms.

Nitric oxide production was quantified via Greiss assay and intracellular staining with DAF-FM diacetate.

6. Endothelial Cell Viability

PAEC were cultured and infected as previously described. Forty-eight hours after infection, PAEC were incubated for 1 hour under 500 μM H₂O₂ in serum-free M199; control groups were incubated in serum-free M199. A concentration of 500 μM H₂O₂ was used as a severe challenge to examine the effects of MnSOD adenoviral infection on cellular viability at high H₂O₂ concentrations. After incubation, 3 μM propidium iodide (PI) was added to each sample and incubated for 8 minutes. Samples were then washed with serum-free M199 and imaged at an exposure of 2500 ms. All fluorescent images were acquired in phenyl-red free media.

7. Superoxide Detection Assay Under Shear

An Immunetics cell adhesion chamber (Cambridge, Mass.) was utilized to determine PAEC behavior under shearing conditions. PAEC at a density of 500,000 cells/slide were cultured on Vitrogen (Cohesion, 0.032 mg/ml) coated glass microscope slides (Surgipath) as previously described. PAEC were exposed to a constant flow rate of 24.4 ml/min (2.2 dynes/cm²) for 1.5 hours. Maintenance of PAEC at 37° C. was achieved by using a fluid reservoir contained in a constant temperature water bath (Precision). Challenged PAEC were exposed to 100 μM H₂O₂ in serum-free and phenyl red free M199; control PAEC were exposed to serum-free and phenyl red free M199 without H₂O₂.

After 1 hour of incubation, 2 μM dihydroethidium was added to the media reservoir. Images were collected at 0, 15, 30, 60, and 90 minutes and a fluorescent image was taken at 90 minutes using a fluorescein filter (FITC) at an exposure of 4500 ms.

8. Data Analysis

Image analysis was conducted with Slidebook 4 Imaging Software (company). Statistical analyses were conducted using the Student's t-Test assuming unequal variance. Values are reported as mean±standard error of the mean (SEM).

### Results

To determine the effects of H₂O₂-induced oxidative stress on PAEC; dihydroethidium (DHE) was used to examine superoxide production.

1. PAEC Produce Superoxide on Exposure to H₂O₂

For control PAEC, 2.6±0.1% of cells under static conditions were found to have fluorescent nuclei. In contrast, H₂O₂ challenged PAEC under similar conditions showed 51.8±1.1% of cells as having fluorescent nuclei.

Manganese superoxide dismutase (MnSOD) overexpression by adenoviral-mediated gene transfer reduced hydrogen peroxide-induced superoxide levels seventy-five percent under static (n=12, p-value less than 0.001) and forty-seven percent under physiological shear (n=4, p=0.03) conditions as assayed by dihydroethidium fluorescence.

The eGFP infected PAEC demonstrated no statistically significant portion of superoxide production that was attributed to adenoviral infection. The viewing field size was standardized at 500 cells per microscopic field at 10× magnification.

2. Enzymatic Pathway Analysis (eNOS Inhibition)

N^{G}-nitro-L-arginine methyl ester (L-NAME), a specific inhibitor of nitric oxide synthase, was used to further examine the enzymatic sources of hydrogen peroxide induced superoxide production within PAEC. Samples exposed to hydrogen peroxide and control samples were cultured in serum-free M199, with 2 μM DHE used as a marker for superoxide production. The non-inhibited controls were found to have 2.0±0.5% cells with fluorescent nuclei suggesting superoxide production, while the control with eNOS inhibition had 2.2±0.5% cells with fluorescent nuclei. The challenged non-inhibited group was found to have 23.8±3.8% cells with fluorescent nuclei, while the samples exposed to hydrogen peroxide and L-NAME was found to have 8.1±1.3%. A sixty percent decrease in fluorescent nuclei was seen with the presence of L-NAME in samples under oxidative stress (p=0.001).

In another set of samples, the control sample cultured with the eNOS inhibitor L-NAME (1 mM) demonstrated 2.9±0.3% cells fluorescent nuclei, while the hydrogen peroxide exposed sample with L-NAME (1 mM) demonstrated 19.3±1.1% of cells with fluorescent nuclei. Thus, in the presence of L-NAME (1 mM), a specific inhibitor of nitric oxide synthase (NOS), there was a sixty percent reduction in cellular superoxide production (n=9, p=0.001).

All challenged sample groups were exposed to 60 μM H₂O₂. Viewing field size was standardized at 500 cells per microscopic field at 10× magnification.

3. PAEC Viability Under Extreme H₂O₂

An increase in PAEC viability infected with MnSOD was determined by propidium iodide. Propidium iodide is actively transported out of living cells and accumulates in dead or dying cells and can be used as a marker for cell viability. Control Less than five percent of PAEC in both control groups were positive for propidium iodide staining. The challenged infected PAEC showed less than fifteen percent positive cells; an increase over control samples. Challenged infected PAEC presented more than ninety percent positive cells and nearly ten percent had ruptured cellular membranes. All severely challenged sample groups were exposed to 500 μM H₂O₂.

4. Apocynin Has Minimal Effects on Superoxide Production

Apocynin (0.5 mM), an inhibitor of NAD(P)H oxidase, had minimal effects on superoxide production. A control sample cultured with the NAD(P)H oxidase inhibitor apocynin (0.25 μM, top) demonstrated 14.2±1.3% cells fluorescent nuclei; while the hydrogen peroxide exposed sample with apocynin (0.25 μM, bottom) demonstrated 35.1±2.0% of cells with fluorescent nuclei.

5. Cyclooxygenase and Xanthine Oxidase Inhibitors

A control sample cultured with the Cyclooxygenase inhibitor flurbiprofen demonstrated 3.9±0.4% cells fluorescent nuclei; while the hydrogen peroxide exposed sample with flurbiprofen demonstrated 46.8±1.4% of cells with fluorescent nuclei.

The control sample cultured with the Xanthine oxidase inhibitor allopurinol demonstrated 4.1±0.4% cells fluorescent nuclei; while the hydrogen peroxide exposed sample with allopurinol demonstrated 35.6±1.3% of cells with fluorescent nuclei.

6. Superoxide Detection Under Shear

Hydroethidium was used to examine superoxide production of infected and non-infected PAEC under H₂O₂ induced oxidative stress. PAEC on glass slides were subjected to laminar flow at 24.4 ml/min (2 dynes/cm²). The circulating fluid contained 2 μM dihydroethidium as a marker for superoxide production.

For infected control PAEC, 10.9±6.0% of cells were found to have fluorescent nuclei. While for the infected challenged PAEC, 26.6±6.5% of cells had fluorescent nuclei suggesting superoxide production. For the control group, 1.6±1.0% of the PAEC were found to have fluorescent nuclei and the cellular membranes are still intact. In the challenged PAEC, 50.3±4.1% of cells were found to have fluorescent nuclei. Superoxide producing cells with white nuclei were observed, with notable rupture of PAEC membranes; in contrast with other PAEC groups. A 47% decrease in fluorescent nuclei was seen with infected stressed samples compared with the non-infected stressed samples (p=0.027). All challenged sample groups were exposed to 100 μM H₂O₂. Viewing field size was standardized at 1000 cells per microscopic field at 10× magnification.

Therefore, hydrogen peroxide induces production of superoxide in PAEC cells under both static and shear conditions. This production contributes to cytotoxicity in these cells. eNOS, rather than NADPH oxidase, is an important enzymatic source of hydrogen peroxide-induced superoxide production in PAEC.

7. Observations

The current study probed the effects of PAEC exposure to hydrogen peroxide. Four major conclusions can be made from this study: 1) Hydrogen peroxide exposure triggers PAEC production of superoxide; 2) these results are conserved under physiological shear; 3) eNOS is a major enzymatic contributor to this production; 4) eNOS is uncoupled with exposure to H₂O₂.

Under static culture, exposure of PAEC to hydrogen peroxide triggered increases in production of superoxide as determined via DHE analysis. A 10-fold increase in the percent of superoxide producing cells between the control and H₂O₂ induced oxidative stressed samples was observed. There is a significant increase (p<0.001) in superoxide production between the control and sample exposed to hydrogen peroxide. This demonstrates the ability of hydrogen peroxide to increase superoxide production in PAEC. Hydrogen peroxide has previously been shown to stimulate superoxide production in arterial smooth muscle cells (SMC) via the NAD(P)H oxidase pathway.

With the addition of genetic modification with the Ad5CMVSOD2 (MnSOD) adenovirus, an 8-fold decrease in the percent of fluorescent cells between infected and non-infected samples groups under H₂O₂ induced oxidative stress were observed. There is an effective decrease in the concentration of superoxide due to the scavenging nature of superoxide dismutase. With adenoviral infection the percent fluorescent cells was significantly decreased with exposure of PAEC to hydrogen peroxide. In the fluorescent PAEC, ethidium bromide was localized in the nuclei of superoxide producing cells, there was also evidence of ethidium bromide association with mitochondria; this was only observed in samples under H₂O₂-induced oxidative stress. The intensity of fluorescence of the mitochondria was significantly lower then the nuclei and both infected and non-infected samples exhibited this behavior. A similar affect was not noted under shear conditions. Cellular membrane integrity was also degraded with hydrogen peroxide exposure; globules of cytoplasm were observed and they occurred only in non-infected stressed samples. These data demonstrated hydrogen peroxide induced superoxide production and a decrease in the effects of this increase in superoxide production with MnSOD adenoviral infection. Other studies have demonstrated nitric oxide stimulation with hydrogen peroxide at low and higher concentrations then the 60 H₂O₂ used in experimentation; also due to the limited length of experimentation no significant increases in NO production were observed as demonstrated through DAF-AM acetate intracellular staining.

To further increase the understanding of superoxide production in PAEC exposed to hydrogen peroxide; the enzymatic pathway for the superoxide production was probed. Previous studies have shown NADPH oxidase (Johnson et al., 2002) and xanthine oxidase (Carter et al., 1994) to be strong contributors to superoxide production. In this study, eNOS was a significant enzymatic source of superoxide in endothelial cells under hydrogen peroxide induced oxidative stress. There a 60% decrease in superoxide production in the presence of L-NAME in non-infected samples exposed to hydrogen peroxide. This production is likely due to the uncoupling of eNOS in the presence of hydrogen peroxide as demonstrated with experimentation using ascorbic acid where superoxide production was almost completely inhibited (p<0.001).

These results combined with the earlier published studies on superoxide production under hydrogen peroxide further the understanding of hydrogen peroxide as an inducer of oxidative stress in PAEC. This pathway was further probed with the use of an eNOS encoded adenovirus that led to increases in nitric oxide production as measured by the Greiss assay. No statistical difference was observed between H₂O₂ challenged PAEC and eNOS infected H₂O₂ challenged PAEC as measured by DHE staining. Cytoplasm leakage was observed in infected challenged samples, suggesting that increases in nitric oxide combined with superoxide lead to increased formation of peroxynitrite and further cellular damage.

Under shear conditions, exposure of PAEC to hydrogen peroxide triggered increases in production of superoxide as seen in static experiments. A 30-fold increase in the percent of superoxide producing cells between the non-infected control and non-infected H₂O₂ induced oxidative stressed samples was observed under physiologic shear. There is a significant increase (p<0.001) in superoxide production between the control and sample exposed to hydrogen peroxide. This demonstrates the ability of hydrogen peroxide to increase superoxide production in PAEC and is similar to the affect seen under static conditions. An increase in superoxide production was found under shear when compared to static experimentation. PAEC phenotype was probed using the Greiss assay for nitric oxide; and as shown in other studies there was a substantial increase in nitric oxide production from the samples under flow in comparison to in vitro cultures (data not shown) (Hon et al., 2000). The increase in superoxide production is likely due to upregulation of cellular processes within the shearing environment as an increase in nitric oxide production was also observed and has been shown in the literature. This increase in nitric oxide combined with superoxide likely will lead to an increase in peroxynitrite.

With the addition of genetic modification with the Ad5CMVSOD2 (MnSOD) adenovirus, a 2-fold decrease in the percent of fluorescent cells between infected and non-infected samples groups under H₂O₂ induced oxidative stress were observed. There is an effective decrease in the concentration of superoxide due to the scavenging nature of superoxide dismutase. With adenoviral infection the percent fluorescent cells was significantly decreased with exposure of PAEC to hydrogen peroxide. There was a decrease in the reduction of the effective superoxide concentration by MnSOD adenoviral infection from static to shear conditions. Under shear, increased numbers of PAEC had increased levels of intracellular superoxide as shown via dihydroethidium assay. This is likely due to the increase in nitric oxide seen under shear allowing for an increase in the formation of peroxynitrite due to the faster rate of combination of nitric oxide and superoxide compared with the rate of superoxide scavenging via superoxide dismutase. Cellular membrane integrity was also degraded with hydrogen peroxide exposure under shear; this was demonstrated by the loss of cellular adhesion to the surface. In non-infected stressed samples, fifty percent of all samples in the group lost more then 25% of the PAEC population within the viewing field. This effect was not observed in stressed samples exposed to hydrogen peroxide or in any control groups.

Previous published reports have shown that adenoviral infection reduces cellular attachment. While this study did not focus directly on this issue, infected stressed samples remained attached to the surface, while the data demonstrates a loss PAEC in non-infected stressed samples under physiologically relevant shear. The MnSOD adenoviral infection provided protection to the PAEC and did not adversely interfere with attachment within this study. These data demonstrates hydrogen peroxide induced superoxide production and a decrease in the effects of this increase in superoxide production with MnSOD adenoviral infection under shear and static culture conditions.

In other studies a corresponding protective effect due to SOD was seen in smooth muscle cells (Li et al., 2001). From this study and the smooth muscle study it appears that gene transfer produces decreased effective superoxide levels, likely due to superoxide scavenging via superoxide dismutase. The protective affect of SOD on PAEC is further documented through the propidium iodide viability assay. There was a statistical difference between the infected and non-infected samples under H₂O₂ induced oxidative stress. There is at least a four-fold increase of non-viable cells between the infected and non-infected groups. Over ninety percent of the cells remain intact; cytoplasm debris were only observed in the H₂O₂ challenged PAEC. This further demonstrates the increased survivability of the infected cells under oxidative stress versus non-infected cells. This is true even with the fact that the H₂O₂ concentration is 400% greater then was used in the hydroethidium experiments under physiologic shear.

From this study, endothelial cells were observed to produce superoxide when exposed to hydrogen peroxide under both static and physiologically relevant shear conditions. Upregulation of superoxide dismutase production with an adenoviral vector led to a protective affect of infected samples when exposed to hydrogen peroxide under static culture conditions. eNOS was found to be a major contributor to superoxide production in hydrogen peroxide induced oxidative stress. The protective effect of MnSOD adenoviral infection was conserved under physiologically relevant shearing conditions.

All of the compositions and/or methods and/or apparatus disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and/or apparatus and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references are specifically incorporated herein by reference.
Association, Heart Disease and Stroke Statistics-2003 Update, American Heart Association: Dallas, Tex., 2002.
Baas and Berk, Circulation Res., 77(1):29-36, 1995.
Beckman et al., Proc. Natl. Acad. Sci. USA, 87(4):1620-1624, 1990.
Blackman et al., Ann. Biomed. Eng., 28(4):363-372, 2000.
Boveris et al., Methods in Enzymology, 349:280-287, 2002.
Cai and Harrison, Circulation Res., 87(10):840-844, 2000.
Carter et al., J. Leukocyte Biology, 55(2):253-258, 1994.
Chandrasekar et al., Biochem. Biophys. Res. Communications, 303(4):1152-1158, 2003.
Corson et al., Circulation Res., 79(5):984-991, 1996.
Cowling et al., J. Biol. Chemistry, 277(8):5719-5724, 2002.
De Keulenaer et al., Circulation Res., 82(10):1094-1101, 1998.
Desai et al., Endothelium, 9(2):89-102, 2002.
Fang et al., Circulation Res., 82(12):1289-1297, 1998.
Fisher et al., Amer. J. Physiology—Lung Cell. Molec. Physiology, 281(3):L529-533, 2001.
Gonzalez-Pacheco et al., Nephrology Dialysis Transplant., 17(3):392-398, 2002.
Harrison et al., J. Renin-Angiotensin-Aldosterone Sys., 4(2):51-61, 2003.
Harrison and Cai, Cardiology Clinics, 21(3):289-302, 2003.
Hohler et al., Histochemistry Cell Biology, 114(1):29-37, 2000.
Hon et al., Artificial Organs, 24(5):387-390, 2000.
Imberti et al, Endothelium, 9(1):11-23, 2002.
Jeremy et al., J. Cardiac Surg., 17(4):324-327, 2002.
Jin et al., Medical Sci. Monitor, 7(4):585-591, 2001.
Johnson et al., Endothelium, 9(3):191-203, 2002.
Kader et al., J. Biomed. Materials Res., 60(3):405-410, 2002.
Kader et al., Tissue Engineering, 6(3):241-251, 2000.
Kimura, Intl. J. Biochem. Cell Biology, 29(3):437-446, 1997.
Kojda and Harrison, Cardiovascular Res., 43(3):562-571, 1999.
Kossila et al., Molec. Therapy: J. Amer. Soc. Gene Therapy, 5(1):87-93, 2002.
Lacy et al., J. Hypertension, 16(3):291-303, 1998.
Li et al., Amer. J. Respir. Cell Molec. Biology, 14(4):398-406, 1996.
Li et al., J. Biol. Chemistry, 276(31):29251-29256, 2001.
McNally et al., Amer. J. Physiology—Heart Circul. Physiology, 285(6):H2290-2297, 2003.
McQuaid and Keenan, Experim. Physiology, 82(2):369-376, 1997.
Miller et al., Circulation Res., 82(12):1298-1305, 1998.
Muller-Glauser et al., Altex, 5(1):34-50, 1988.
Nerem et al., Amer. J. Med. Sciences, 316(3):169-175, 1998.
Nerem, J. Biomech. Engineering, 114(3):274-282, 1992.
Pryor and Squadrito, Amer. J. Physiology, 268(5 Pt 1):L699-722, 1995.
Rao et al., Nucleic Acids Research, 21(5):1259-1263, 1993.
Rao et al., Oncogene, 8(10):2759-2764, 1993.
Saavedra et al., Circulation Res., 90(3):297-304, 2002.
Saiki et al., J. Natl. Cancer Institute, 77(6):1235-1240, 1986.
Schiffrin et al., Amer. J. Hypertension, 15(10 Pt 2):115S-122S, 2002.
Sodhi et al., Amer. J. Physiology—Heart Circul. Physiol., 284(3):H846-852, 2003.
Strawn and Ferrario, Curr. Opin. Lipidology, 13(5):505-512, 2002.
Tarpey and Fridovich, Circulation Res., 89(3):224-236, 2001.
Tarpey et al., Circulation Res., 84(10):1203-1211, 1999.
Tazawa et al., J. Pharmacol. Experim. Therapeutics, 288(2):898-904, 1999.
Tesfamariam and Cohen, Amer. J. Physiology, 263(2 Pt 2):H321-326, 1992.
Tesfamariam, Free Radical Biol. Medicine, 16(3):383-391, 1994.
True et al., Amer. J. Physiology—Lung Cell. Molec. Physiology, 279(2):L302-311, 2000.
Ullian et al., Amer. J. Physiology, 273(4 Pt 1):C1241-1249, 1997.
Vanizor et al., Diabetes Res. Clinical Prac., 54(1):33-39, 2001.
von Harsdorf et al., Circulation, 99(22):2934-2941, 1999.

1. A method for screening for a candidate drug to relieve oxidative stress in an endothelial cell comprising:
(a) contacting an endothelial cell with DHE;
(b) contacting the endothelial cell with a candidate substance;
(c) subjecting the endothelial cell to physiologic shear; and
(d) comparing the amount of DHE oxidized by the cell with the amount of DHE oxidized by a cell not contacted with the candidate substance.

2. The method of claim 1, wherein the cell is contacted with about 1.0 μM to about 5.0 μM of DHE.

3. The method of claim 1, further comprising exposing the endothelial cell to hydrogen peroxide.

4. The method of claim 3, wherein the cell is exposed to about 30 μM to about 100 μM of hydrogen peroxide.

5. The method of claim 4, wherein the cell is contacted with about 1.0 μM to about 5.0 μM of DHE.

6. The method of claim 1, further comprising contacting the cell with an inhibitor of eNOS, NADPH, endothelial nitric oxide synthase, NADH/NADPH-oxidase, cyclooxygenase, or xanthine oxidase.

7. A method of identifying a target or pathway involved in endothelial cell function comprising:
(a) providing an endothelial cell;
(b) contacting said endothelial cell with (i) a first substance that acts as an indicator of a first cell function in said endothelial cell and (ii) a modulator of a known enzyme, receptor, transporter, signaling molecule, or transcription factor;
(c) subjecting said endothelial cell to physiologic shear; and
(d) assessing said first cell function;
wherein a change in said first cell function in the presence of said modulator, as compared to said first cell function in the absence of said modulator, identifies said inhibitor as acting on a target or pathway involved in said first cell function.

8. The method of claim 7, wherein said first cell function is NO production.

9. The method of claim 8, wherein first substance is DHE, and said method further comprises contacting said endothelial cell with H₂O₂.

10. The method of claim 7, wherein said modulator is an agonist of said first cell function.

11. The method of claim 7, wherein said modulator is an antagonist of said first cell function.

12. The method of claim 7, wherein said modulator acts on an enzyme.

13. The method of claim 12, wherein said modulator is an antagonist of eNOS, NADPH, endothelial nitric oxide synthase, NADH/NADPH-oxidase, cyclooxygenase, or xanthine oxidase.

14. The method of claim 7, wherein said modulator acts on a receptor.

15. The method of claim 9, wherein H₂O₂ is present at about 30 μm to about 100 μm.

16. A method of inhibiting NO production in an endothelial cell comprising contacting said cell with an inhibitor of eNOS, NADPH, endothelial nitric oxide synthase, NADH/NADPH-oxidase, cyclooxygenase, xanthine oxidase.

17. The method of claim 16, wherein said endothelial cell is located in a living organism.

18. The method of claim 17, wherein said living organism is suffering from a disease state.

19. The method of claim 18, wherein said disease state is diabetes or cardiovascular disease.

20. The method of claim 16, wherein said inhibitor is L-NAME or L-NNA.

21. The method of claim 17, wherein said living organism is a human.
